(19) United States
(12) Patent Application Publication
(10) Pub. No.: US 2018/0289775 A1
(43) Pub. Date: Oct. 11, 2018

(54) MODIFIED COLLAGEN COMPOSITIONS FOR MODULATION OF JNK

(71) Applicant: Southwest Technologies, Inc., North Kansas City, MO (US)

(72) Inventors: Edward I. Stout, North Kansas City, MO (US); Chandan Sen, Columbus, OH (US)

(21) Appl. No.: 15/946,943

(22) Filed: Apr. 6, 2018

Related U.S. Application Data
(60) Provisional application No. 62/482,308, filed on Apr. 6, 2017.

(51) Int. Cl. A61K 38/39; A61K 47/42; A61K 9/00

(52) U.S. Cl. CPC A61K 38/39; A61K 9/0014; A61K 9/0019; A61K 47/42

(57) ABSTRACT

Methods and compositions for modulating JNK activity and stimulating efferocytosis in a cell or patient are described. Therapeutic modified collagens comprising a plurality of proteins characterized by Table 1 for use as JNK modulators are also described.

### CROSS-REFERENCE TO RELATED APPLICATIONS

The present application claims the priority benefit of U.S. Provisional Patent Application Ser. No. 62/482,308, filed Apr. 6, 2017, entitled ROLE OF THE MODIFIED COLLAGEN GEL IN THE JNK PATHWAY, incorporated by reference in its entirety herein.

### FEDERALLY SPONSORED RESEARCH OR DEVELOPMENT

This invention was made with government support under GM077185 awarded by National Institutes of Health. The government has certain rights in the invention.

### BACKGROUND OF THE INVENTION

### Field of the Invention

The present invention relates to compositions, methods, and treatment protocols for modulation of the INK pathway using a therapeutic modified collagen gel.

### Description of Related Art

The c-Jun N-terminal kinases (JNKs), also called stress-activated protein kinases (SAPKs), are among the major signaling cassettes of the mitogen-activated protein kinase (MAPK) signaling pathway. These enzymes function in the control of a number of cellular processes, including proliferation, embryonic development, and apoptosis. The JNK pathway is sometimes referred to as a cellular “death” pathway. Activation of the JNK pathway has been documented in a number of disease settings, and much focus has been on inhibition or interruption of JNK signaling as a promising approach for combatting disorders related to INK signaling. However, apoptosis is also an important step of healing. Accordingly, there is a need in the art for modulators of the INK pathway. In addition, there is a need for therapeutic compositions comprising one or more JNK modulators, as well as to methods for treating conditions in animals which are responsive to such modulators. The present invention fulfills these needs, and provides further related advantages.

### SUMMARY OF THE INVENTION

Described herein are methods for regulating expression and modulating (i.e., increasing or decreasing) levels of JNK expression and activity. The modulation of JNK activity includes inhibitory or stimulatory effects. Preferably, the invention is concerned with inducing JNK activity, specifically JNK activation in macrophages, and increasing efferocytosis of apoptotic cellular components. The invention finds use in regulating JNK activation and modulating JNK-mediated signal transduction in various cellular pathways for both therapeutic use and clinical study. In one or more embodiments, JNK modulators of the invention are used as promoting agents to selectively upregulate the JNK pathway.

In one aspect, methods of modulating JNK activity in a cell expressing JNK are described. The methods generally comprise contacting the cell with a therapeutically effective amount of a therapeutic modified collagen, as described in detail herein. In one aspect, healthy cells are targeted by the methods, and specifically macrophages. In one aspect, healthy cells at the site of wound or tumor are targeted.

In another aspect, methods of modulating JNK activity in a patient in need thereof are described. The methods generally comprise administering a therapeutically effective amount of a therapeutic modified collagen composition to the patient, such that the modified collagen increases JNK expression in the patient. Such methods are useful in the treatment of JNK-mediated disorders related to under-expression of JNK, such as inflammatory, autoimmune, cardiovascular, ischemic, neurodegenerative, or metabolic conditions, infections, diabetes, or cancer.

JNK modulators comprising a therapeutic modified collagen comprising a plurality of proteins characterized by Table 1 are particularly useful in the present invention. The JNK modulator could be formulated as a powder, tablet, suspension, ointment, hydrogel, etc. depending upon the desired route of administration.

Other aspects described herein relate to stimulating efferocytosis in a cell or subject, comprising contacting or administering a therapeutically effective amount of a JNK modulator according to embodiments of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. (FIG.) 1A shows the results from flow cytometry analysis of the PVA sponges harvested from the C57bl/6 mice at on day 3 post-implantation, including a plot of all wound inflammatory cells subjected to F4/80 analysis and histograms showing the F4/80-FITC signals on the x-axis;

FIG. 1B shows the results from flow cytometry analysis of the PVA sponges harvested from the C57bl/6 mice at on day 7 post-implantation, including a plot of all wound inflammatory cells subjected to F4/80 analysis and histograms showing the F4/80-FITC signals on the x-axis;

FIG. 1C shows a graph of F4/80+ cells quantified from the gated cell populations shown in FIG. 1A & 1B. Data are mean±SEM (n=3); *p<0.05 compared to cells harvested from MCG-untreated PVA sponges;

FIG. 2 shows quadrant dot plots, histograms, and graphs of immunostained cells harvested at day 3 and analyzed by flow cytometry;

FIG. 3 shows quadrant dot plots, histograms, and graphs of immunostained cells harvested at day 7 and analyzed by flow cytometry;

FIG. 4 shows graphs of RT-PCR analysis of mRNA expression of IL-4 (A) and IL-10 (B), and ELISA analysis of protein expression of IL-4 (C) and IL-10 (D), in cells collected from MCG-treated sponges, as compared to cells collected from untreated sponges (control);

FIG. 5 shows the results from ELISA analysis of IL-10 and VEGF protein release in human acute monocytic leukemia cell-line THP-1 cultured and differentiated to macrophages, followed by direct treatment with modified collagen gel (MCG), or left untreated (control);

FIG. 6 shows the results for day 3 wound cells harvested from the sponges and subjected to an efferocytosis assay, including (A) representative images showing harvested MCG-treated macrophages (green, F4/80) cultured with apoptotic thymocytes (red, CMTMR cell tracker), and counterstained with DAPI (nuclear, blue); and (B) a graph of the efferocytosis index of apoptotic thymocytes engulfed by macrophages, calculated as total number of apoptotic cells engulfed by macrophages in a field of view divided by total number of macrophage presented in the view. Data are mean±SEM (n=7-8); *p<0.05 compared to control;

FIG. 7A shows a graph of the miR-21 expression in mouse inflammatory cells collected from MCG-treated sponges at day 3 post-implantation, presented as % change compared to untreated cells. Data are mean±SEM (n=4); *p<0.05 compared to control;

FIG. 7B shows a graph of IL-10 production in miR-000-zip or miR-21-zip cells after treatment with MCG. The miR-21 zip cells show a significant attenuation in miR-21 expression. Data are mean±SEM (n=4); *p<0.05 compared with MCG untreated miR-000-zip (control) cells; †p<0.05 compared with MCG treated miR-000-zip cells; and

FIG. 7C shows a graph of IL-10 production in differentiated THP-1 cells after treatment with 420119 JNK Inhibitor II and MCG treatment. Data are mean±SEM (n=4); *p<0.05 compared with MCG untreated (control) cells; †p<0.05 compared with MCG-treated and JNK inhibitor untreated cells.

### DETAILED DESCRIPTION

The present invention is concerned with compositions having activity as modulators of the JNK pathway. Such compositions have utility in the treatment of a wide variety of conditions that are responsive to modulation of the JNK pathway. Since JNKs comprise a central node in the inflammatory signaling network, hyperactivation of JNK signaling is a common finding in a number of disease states including cancer, inflammatory, and neurodegenerative diseases. However, the JNK pathway is also critical to clearance of apoptotic cells leading to resolution of inflammation and healing. For example, increased apoptotic cell burden at a wound site exacerbates sustained inflammation. Further, the development of atherosclerotic plaque leading to coronary artery disease has been linked to over-retention of apoptotic smooth muscle cells. Efferocytosis (i.e., engulfment of apoptotic cells by macrophages) has also been determined as a signaling cue that drives the wound healing process from the pro-inflammatory M1 macrophages to the reparative M2 phenotype that is essential for the resolution of inflammation. Thus, promoting efferocytosis through selective inducement of the JNK appears likewise critical to promoting anti-inflammatory effects under the right circumstances.

Methods described herein are concerned with modulating JNK activity in a cell expressing JNK, comprising contacting the cell with an therapeutically effective amount of a therapeutic modified collagen gel. The methods herein are also concerned with treating or preventing a condition responsive to JNK pathway modulation, comprising administering to a patient in need thereof a therapeutically effective amount of therapeutic modified collagen gel. Exemplary conditions include any condition that is responsive to JNK pathway modulation, such as inflammatory, autoimmune, cardiovascular, ischemic, neurodegenerative, or metabolic conditions, infections disease, cancer, and the like.

The ability of the therapeutic modified collagen gel to modulate inducible JNK activation also has utility as an anti-inflammatory agent, or in the inducement of cell apoptosis or other forms of cell death, efferocytosis, as a neuroprotective agent, cancer therapeutic agent, and in treating complications from diabetes. Methods of modulating inducible JNK activation in a patient are thus described, wherein the administered modified collagen gel induces JNK activation, efferocytosis, and resolution of inflammation in the patient.

As used herein, the term “therapeutically effective” refers to the amount and/or time period that will elicit the biological or medical response of a tissue, system, animal, or human that is being sought by a researcher or clinician, and in particular elicit some desired therapeutic effect. For example, in one or more embodiments, therapeutically effective amounts and time periods are those that induce activation of JNK (and increase related efferocytosis activity). One of skill in the art recognizes that an amount or time period may be considered “therapeutically effective” even if the condition is not totally eradicated but improved partially.

The methods herein involve “therapeutic” modified collagens or collagen gels, which are defined herein as allogeneic or xenogeneic collagen gel compositions originating externally (as contrasted with the patient's own collagen) and applied or administered for a therapeutic purpose to treat a condition in the patient. Exemplary modified collagen gels include Stimulen™ (Southwest Technologies, Inc., North Kansas City, Mo.). In general, the modified collagen gel comprises a dispersion of modified collagens in a glycerine or other biocompatible matrix. In one or more embodiments, the collagen gel comprises modified collagen of long and short polypeptides dispersed in an aqueous matrix comprising (consisting essentially or even consisting of) water and glycerine. The collagen gel comprises at least about 2% by weight modified collagen, preferably from about 2% to about 75% by weight modified collagen, more preferably from about 25 to about 75%, and in some cases, preferably about 52% by weight modified collagen, based upon the total weight of the composition taken as 100% by weight. The collagen gel comprises at least about 15% by weight glycerine, preferably from about 15% to about 65% by weight glycerine, more preferably from about 25% to about 65%, and in some cases, preferably about 45% by weight glycerine, based upon the total weight of the composition taken as 100% by weight.

In some embodiments, the modified collagen can first be provided in a dry (powdered form), which can then be dispersed into a matrix carrier, such as glycerine and/or water before use if desired. Direct use of the dry powdered form of the therapeutic modified collagen is also contemplated herein. In one or more embodiments, the modified collagen is a hydrolyzed bovine collagen. In one or more embodiments, the collagen comprises primarily Type I and Type III collagens (and mainly Type I, more preferably at least about 75% Type I, even more preferably at least about 90%).

Specific proteomic components of the preferred therapeutic modified collagen for use in the invention are provided in the Table below.

TABLE 1
Proteomic Analysis of MCG Components*

| | | | | | Number of | |
|---|---|---|---|---|---|---|
| | | | Unigene | Mass | significant | |
| Sl. No | Description | Accession | ID | (Da) | sequences | Score |
| 1 | Hemoglobin | HBB_BOVIN | Bt.23726 | 16001 | 7 | 685 |
| | subunit beta | | | | | |
| 2 | Carbonic | CAH2_BOVIN | Bt.49731 | 29096 | 10 | 650 |
| | anhydrase 2 | | | | | |
| 3 | Collagen alpha- | CO1A1_BOVIN | Bt.23316 | 139880 | 3 | 321 |
| | 1 (1) chain | | | | | |
| 4 | Hemoglobin | HBA_BOVIN | Bt.10591 | 15175 | 5 | 319 |
| | subunit alpha | | | | | |
| 5 | Peroxire doxin-2 | PRDX2_BOVIN | Bt.2689 | 22217 | 5 | 308 |
| 6 | Alpha-1- | A1AT_BOVIN | Bt.982 | 46417 | 2 | 220 |
| | antiproteinase | | | | | |
| 7 | Serpin A3-7 | SPA37_BOVIN | Bt.55387 | 47140 | 3 | 161 |
| | | | Bt.92049 | | | |
| 8 | Collagen alpha- | CO3A1_BOVIN | Bt.64714 | 93708 | 2 | 147 |
| | 1(III) chain | | | | | |
| 9 | Collagen alpha- | CO1A2_BOVIN | Bt.53485 | 129499 | 2 | 103 |
| | 2(I) chain | | | | | |
| 10 | Serpin A3-3 | SPA33_BOVIN | Bt.55387 | 46411 | 2 | 85 |
| | | | Bt.92049 | | | |
| 11 | Actin, aortic | ACTA_BOVIN | Bt.37349 | 42381 | 2 | 79 |
| | smooth muscle | | | | | |

Top ten most abundant proteins as detected using proteomics analysis has been presented.
Two unique peptides from one protein having a -b or -y ion sequence tag of five residues or better were accepted.
*From Elgharably et al., A modified collagen gel enhances healing outcome in a preclinical swine model of excisional wounds, 21 Wound Repair and Regeneration 473-481 (May-June 2013), incorporated by reference herein. See also, U.S. 2018/0000905, filed Jun. 29, 2017, incorporated by reference herein in its entirety.

Compositions of such therapeutic modified collagens and collagen gels have surprisingly been found to be useful in modulation of the JNK pathway, and in treating conditions responsive to modulation of the JNK pathway. In general, the methods comprise applying or administering a therapeutically effective amount of the composition to a patient in need thereof. The method may involve application of the composition to the site of a wound or infection for a therapeutically effective period of time, or injection into the patient or other suitable administration to the patient. In one or more embodiments, the composition is applied as a dressing to the wound, infection, or tumor site. The composition and/or dressing may be changed periodically, wherein a fresh amount of composition is applied to the site. Additional physiologically-acceptably non-occlusive dressings, tape, gauze, bandages, combinations thereof, and the like may be used in conjunction with the composition, according to standard wound care protocols. In one or more embodiments, a therapeutically effective amount refers to application of the modified collagen gel composition to the site to provide a light coating (e.g., 1/16 inch) up to about ⅛ inch of the composition or more, over the wound. The composition can be changed or re-applied daily, or multiple times per day. Likewise, the composition can be applied every other day, every three days, etc. It is noted that although conventional treatment protocols may call for packing deep wounds, it is not necessary to fill a deep wound cavity with the modified collagen gel, and the wound, infection, or tumor site surfaces can simply be coated with the modified collagen gel, followed by packing the site with a passive dressing to maintain pressure and prevent the modified collagen gel composition from being inadvertently wiped away from the wound, infection, or tumor site. Those skilled in the art will appreciate that treatment protocols can be varied depending upon the type of site, healing status, and preference of the medical practitioner.

In one or more embodiments, the modified collagen may be formulated for parenteral injection into the patient including subcutaneous, intramuscular, intravenous, intraperitoneal, intracardiac, intraarticular, or intracavernous injection, depending upon the disease or condition to be treated by modulation of the JNK pathway. The formulation may be directedly injected into the target site. For example, the modified collagen composition in gel form may be injected via a large bore needle into the target site of the patient (e.g., arthritic joint). Alternatively, a suspension of the powdered composition may be prepared, e.g., in aqueous solution, and administered via injection. Other forms of administration include systemic (indirect) administration, and the like.

Advantageously, the compositions, methods, and treatment protocols can consist of use of only the collagen gel in modulation of JNK activity. In other words, no other adjunctive therapy is required to initiate or promote healing. As such, in some embodiments, the only therapeutic or “active” agent used in treating the wound or condition is preferably the therapeutic modified collagen gel composition. No other antibacterial compositions, ointments, hydrogels, therapeutic dressings, and the like are needed, and can preferably be avoided under typical circumstances. Notwithstanding the foregoing, it will be understood that the methods and treatment protocols would still encompass the use of passive wound care items, such as non-occlusive bandages and gauze, etc. that can be used to cover the treated wound or inflammatory condition once the modified collagen gel has been applied or administered.

In one or more embodiments, the methods are effective for inducing activation of the JNK pathway, activating efferocytosis, and/or resolving inflammation. The composition actively promotes the macrophage anti-inflammatory M2 phenotype via promoting the efferocytosis-JNK-miR-21 pathway. Activation of the JNK pathway yields treated cells or wound sites having improved efferocytosis, a significant induction in miR-21 expression, and decreased inflammation. Thus, methods of the invention relate to modulating the JNK pathway, and specifically inducing JNK, which is defined as activating, promoting, upregulating, stimulating, augmenting, and/or mediating activation of JNK expression in the treated cells.

Methods of the invention also relate to use of the modified collagen gel as a JNK activator, which may be useful for therapeutic purposes and/or for clinical study of the JNK pathway and the effect of other pharmacological or biological modulators on the pathway and related conditions. For example, cells could be incubated in the presence of the JNK activator as well as candidate compound to ascertain the effects of the candidate compound on JNK activity.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Materials and Methods

### Polyvinyl Alcohol (PVA) Sponge Implantation Model

Sterile PVA sponges (circular, 8 mm diameter) were subcutaneously implanted on the back of 8-12 week old C57BL6 mice under anesthesia induced by isofluorane inhalation. The dorsal area of each animal was shaved and cleaned with betadine, and a midline incision (1 cm) was created with a scalpel. Two small subcutaneous pockets were created by blunt dissection, and two PVA sponges containing either MCG or saline (control) were inserted into each pocket. The MCG used was Stimulen™ gel by Southwest Technologies Inc. (North Kansas City, Mo.). The incisions were closed with sutures (5-0 Surgiline™) and the animals were returned to clean cages for monitoring of recovery. Harvesting of the PVA sponges was done 3 and 7 days post-implantation. The animals were euthanized by CO₂ inhalation, followed by removal of all sponges with forceps and placing them in sterile saline. Repeated compression of the sponges in saline resulted in a wound cell suspension which was then filtered with a 70 μm nylon cell strainer (Falcon) to remove all sponge debris, followed by hypotonic lysis with ice cold deionized water to remove the red blood cells.

### Immunostaining and Flow Cytometry

Markers used to identify monocyte/macrophage subsets included: FITC-F4/80 (Serotec) and PE-TLR4, PE-CD16/32, PE-CD11c, PE-CD40, PE-CD23, PE-CD163, and PE-dectin1 (eBioscience). Cells were surface-stained for 60 minutes on ice in staining buffer (1× DPBS/1% BSA). The monocytes were then gated based on forward (FS) and side scatter (SS) characteristics with at least 20,000 gated events recorded using BD FACS Calibur flow cytometry (BD Biosciences) and CellQuest software.

### Cell Culture, Differentiation, and Treatment

The human acute monocytic leukemia cell-line THP-1 were cultured and differentiated to macrophages using PMA treatment (20 ng/ml, 48 h). The differentiated cells were then treated with MCG (100 mg/ml; 72 h). To treat differentiated cells with MCG, a stock solution of MCG was first prepared by dissolving 1 g of MCG in 1 mL of culture media, followed by the addition of 100 μL MCG stock solution to culture plates containing the cells in 900 μL. IL-10 and VEGF protein released from THP-1 differentiated human macrophages was measured by ELISA. The cells were also subjected to treatment with pharmacological JNK inhibitor (420119 JNK Inhibitor II, 20 μM). Data are mean±SEM (n=4); *p<0.05 compared to cells harvested from untreated THP-1 cells.

Isolation of RNA, Reverse Transcription and Quantitative RT-PCR (qRT-PCR)

Wound inflammatory cells on d3 were harvested from MCG treated PVA sponges subcutaneously implanted in C57bl/6 mice. The wound cells were harvested from the sponges, and analyzed for gene expression of IL-4 and IL-10 mRNA. Data are mean±SEM (n=3-4); *p<0.05 compared to compared to cells harvested from untreated PVA sponges. Total RNA was extracted using the mirVana RNA Isolation Kit (Ambion, Austin, Tex.), according to the manufacturer's instructions. mRNA was quantified by real-time or quantitative PCR assay using the dsDNA binding dye SYBR Green I. For determination of miR expression, specific TaqMan assays for miRs and the TaqMan Micro-RNA Reverse Transcription Kit were used, followed by real-time PCR using the Universal PCR Master Mix (Applied Biosystems, Foster City, Calif.).

### Enzyme-Linked Immunosorbent Assay (ELISA)

The wound cells were harvested from the sponges and analyzed for protein expression using ELISA. For measurement of cytokines produced by macrophages, cells were seeded in 12-well plates and cultured in RPMI 1640 medium containing 10% heat-inactivated bovine serum for 24 h under standard culture conditions. After 24 h, the culture media was collected, and cytokine levels were measured using ELISA. Data are mean±SEM (n=3-4); *p<0.05 compared to compared to cells harvested from untreated PVA sponges.

### Apoptotic Cell Clearance (Efferocytosis) Assay

Mouse macrophages that infiltrated MCG-treated PVA sponges were isolated and seeded into 6-well plates. Apoptosis in mouse thymocytes was induced and the efferocytosis assay was performed. Representative images showing harvested MCG-treated macrophages (green, F4/80) cultured with apoptotic thymocytes (red, CMTMR cell tracker). Cells were counterstained with DAPI (nuclear, blue). The efferocytosis index of apoptotic thymocytes engulfed by macrophages was calculated as total number of apoptotic cells engulfed by macrophages in a field of view divided by total number of macrophage presented in the view. Data are mean±SEM (n=7-8); *p<0.05 compared to control.

Stable Knockdown of miR-21 in THP-1 Cells

THP-1 cells with stable knockdown of miR-21 were generated using lenti-miR-000-zip or lenti-miR-21-zip vectors and puromycin selection. Cells with stable knockdown of miR-21 expression were then differentiated to macrophages using PMA treatment.

### Statistics

In vitro data are reported as mean±SD of three to eight experiments as indicated in the respective figure legends. Student's t test (two-tailed) was used to determine significant differences. Comparisons among multiple groups were tested using ANOVA, and p<0.05 was considered statistically significant.

### Results

Increased Macrophage Infiltration at Wounds Treated with MCG

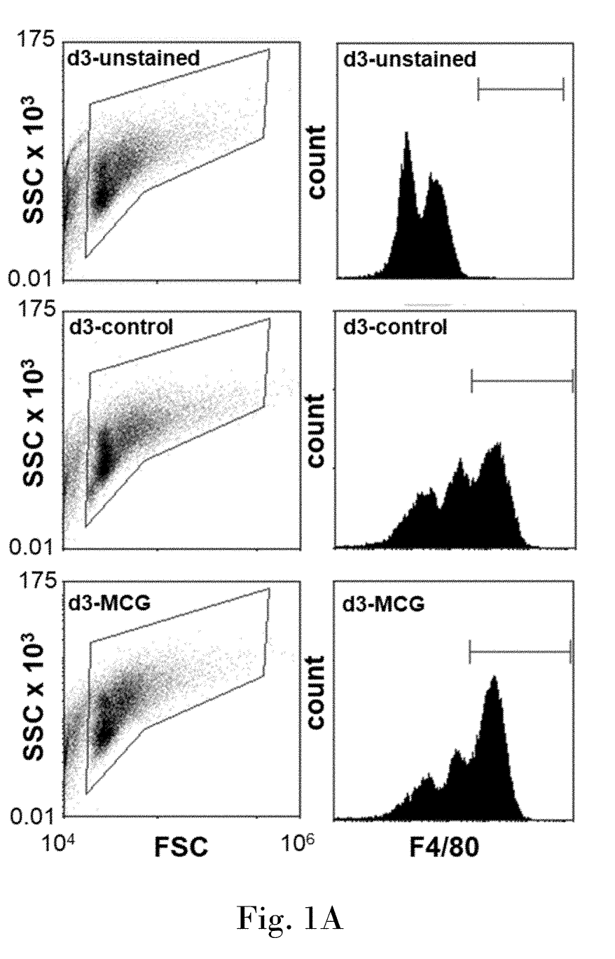

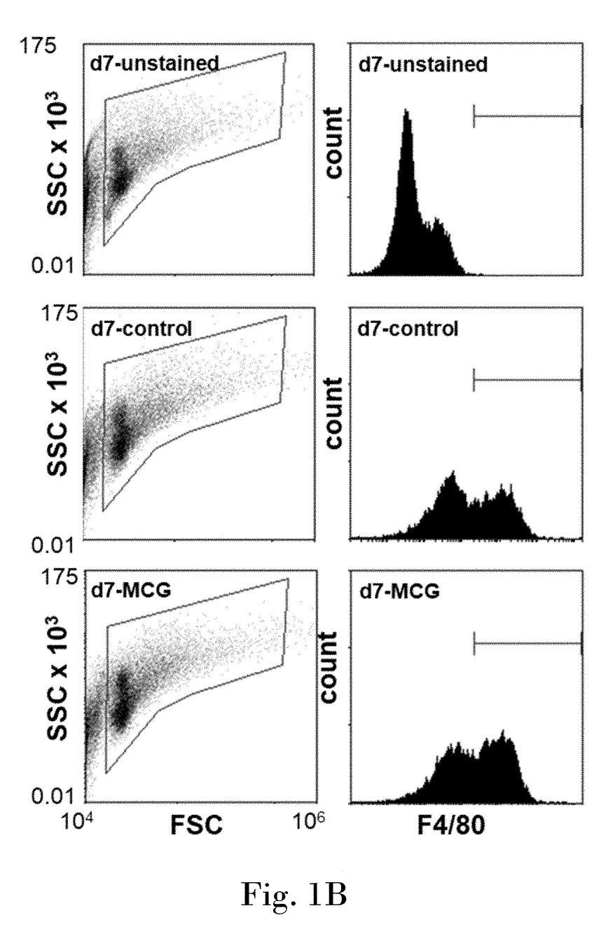

Circulating monocytes recruited to wounded tissues differentiate to macrophages that are critical in orchestrating the inflammatory and subsequent repair process at the wound-site. To determine whether MCG treatment affects the macrophage abundance at the wound-site during early and late inflammatory phases, PVA sponges soaked in MCG solution (2.5 g/ml) were implanted in subcutaneous wounds. The wound cell populations that infiltrated the subcutaneously implanted sponges were collected on days 3 and 7 post-wounding (PW). The total cell population immunostained with FITC conjugated F4/80, a murine macrophage marker, were analyzed using flowcytometry. At both early (d3 PW) and late (d7 PW) inflammatory phases, as shown in FIG. 1, MCG increased macrophage infiltration at the wound-site. MCG treated wounds displayed significantly higher abundance of F4/80⁺ macrophages as compared to untreated wounds (FIG. 1).

### Polarization of Wound Macrophages to Reparative M2 Phenotype in Response to MCG

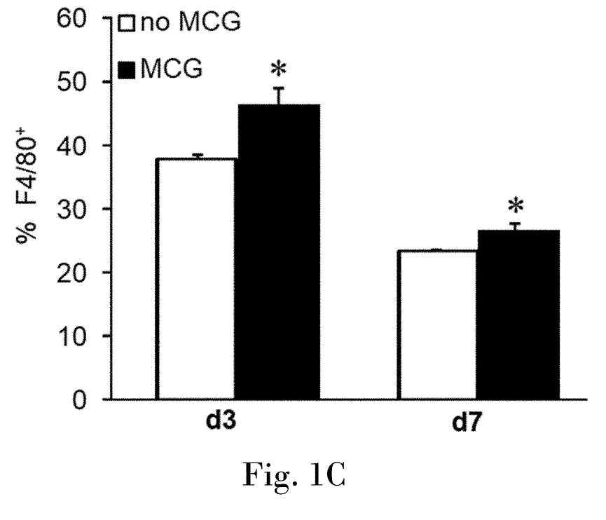

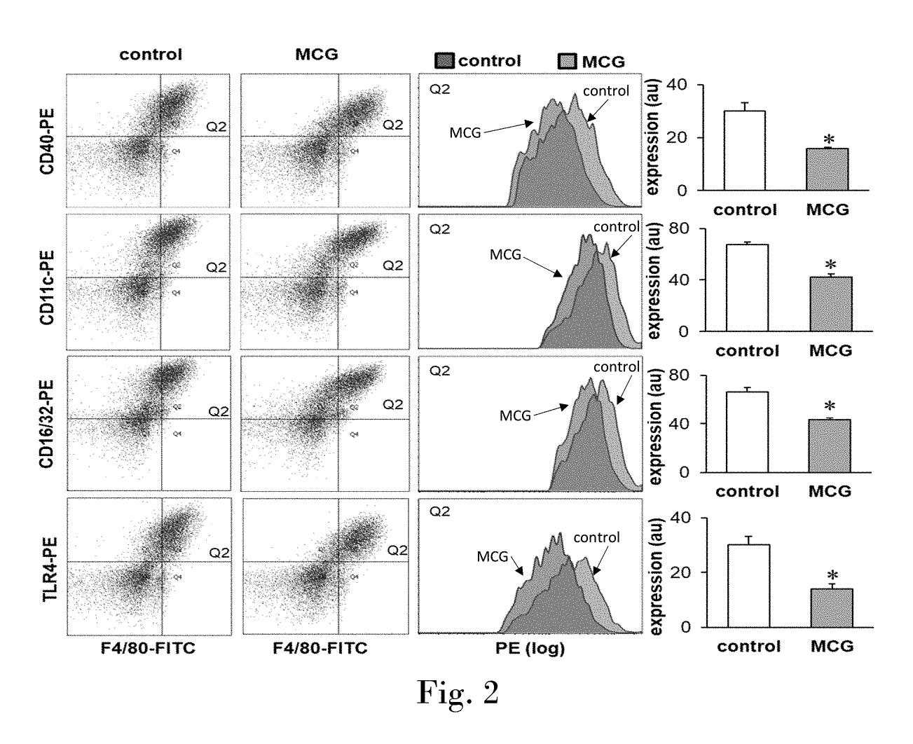

Macrophages have diverse roles in the host inflammatory process, and when induced by certain environmental cues, acquire a distinct functionally polarized proinflammatory (M1) or anti-inflammatory (M2) phenotypes. While the M1 macrophages have microbicidal properties and are predominant in the earlier phase of inflammation, the reparative M2 phenotype is essential for the resolution of inflammation and is predominant in the later phase of inflammation. To determine whether MCG played a role in macrophage polarization, wound cell infiltrate in PVA sponges were dual stained F4/80-FITC (green) and PE-conjugated M1 (CD40, CD11c, CD16/32,TLR-4) or M2 (dectin-1, CD163, CD23) markers (red). The quadrant dual positive for F4/80 and M1/M2 markers were considered in flowcytometry analysis (FIGS. 2-3). As seen in FIG. 2, MCG attenuates M1 polarization of wound macrophage in the early inflammatory phase. The cells were immune-stained using PE tagged M1 markers and co-immunostained with FITC conjugated F4/80 and subjected to flow cytometry analysis. Representative quadrant dot plots and histograms of FITC+PE+ dual positive cells (from quadrant 2 of the dot plot) cells have been shown. Quantitative analysis of the expression (mean fluorescence intensity, MFI) of the double positive cells is expressed as bar graphs for individual M1 marker. Data are mean±SEM (n=3); *p<0.05 compared to cells harvested from untreated PVA sponges. As shown in FIG. 3, there was increased wound macrophage M2 polarization in response to MCG during the late inflammatory phase. Representative quadrant dot plots and histograms of FITC⁺PE⁺ dual positive cells (from quadrant 2 of the dot plot) cells have been shown in FIG. 3. Quantitative analysis of the expression (mean fluorescence intensity, MFI) of the double positive cells is expressed as bar graphs for individual M2 marker. Data are mean±SEM (n=3); *p<0.05 compared to cells harvested from untreated PVA sponges. Based upon this data, significantly lower expression of M1 surface markers in early inflammatory phase and higher expression of M2 markers at late inflammatory phase by MCG are seen, indicating a shift in wound macrophage polarization to an anti-inflammatory, reparative phenotype in the late inflammatory phase (FIG. 2-3).

### Upregulation of Anti-Inflammatory IL-10 in MCG-Treated Wound Cells and Cultured Macrophages

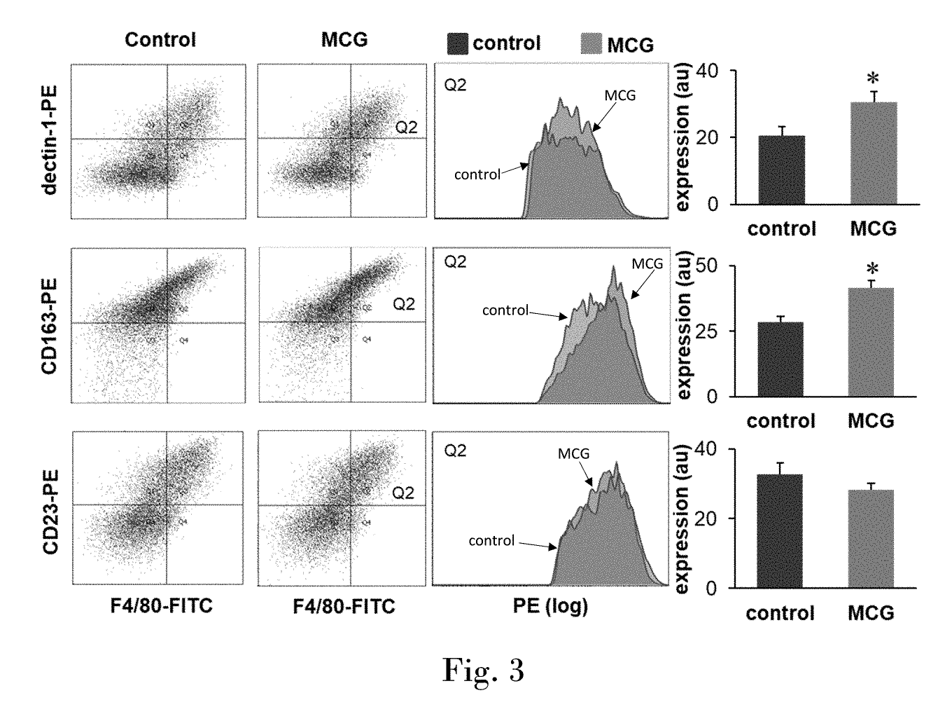

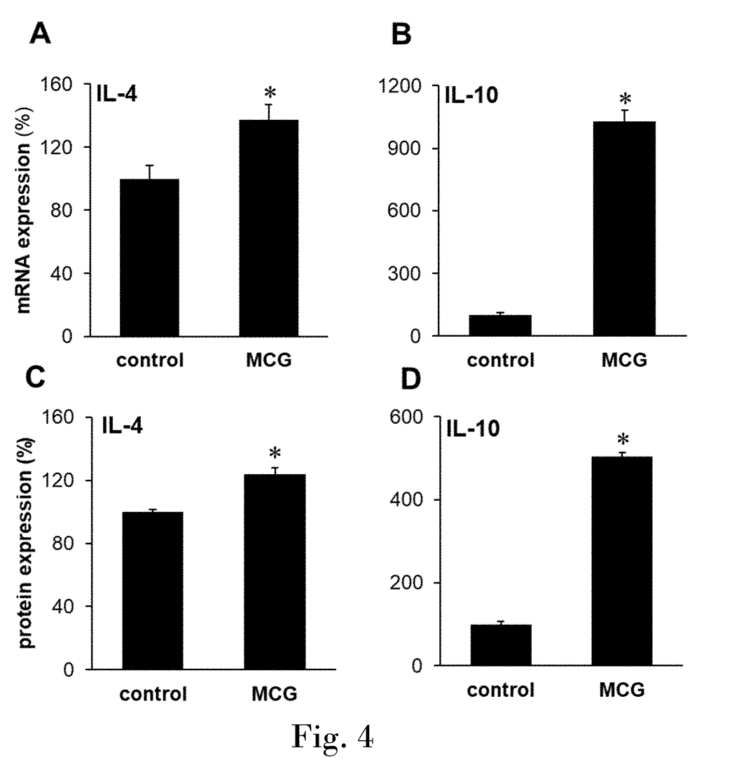

IL-10, also known as human cytokine synthesis inhibitory factor (CSIF) is a cytokine with anti-inflammatory properties. Alternatively, activated M2 macrophages produce copious amount of IL-10 which helps in resolution of inflammation and promotes angiogenesis. To determine whether MCG promoted anti-inflammatory mileu at the wound-site, the mRNA expression of IL-4 (FIG. 4A) and IL-10 (FIG. 4B), two important anti-inflammatory cytokines, were quantified in inflammatory cells derived from MCG-treated wounds. Both IL-4 and IL-10 were strongly upregulated at wound-site at early inflammatory (d3 PW) phase. Accordingly, a strong induction in IL-4 and IL-10 protein was noted in wound inflammatory cells (FIG. 4C). To test a direct effect of MCG on macrophage IL-10 production, differentiated THP-1 derived macrophages were utilized. Measurement of protein by ELISA demonstrated a significant induction in IL-10 protein by THP-1 macrophages following 24 h treatment with MCG confirming a direct action of MCG on macrophages in IL-10 production (FIG. 5A). Vascular endothelial growth factor (VEGF), a potent angiogenic factor secreted by M2 macrophages was also significantly upregulated (FIGS. 5B and 5C) by MCG in THP-1 macrophages.

### MCG Promotes Macrophage Anti-Inflammatory M2 Phenotype via Promoting Efferocytosis-JNK-miR-21 Pathway

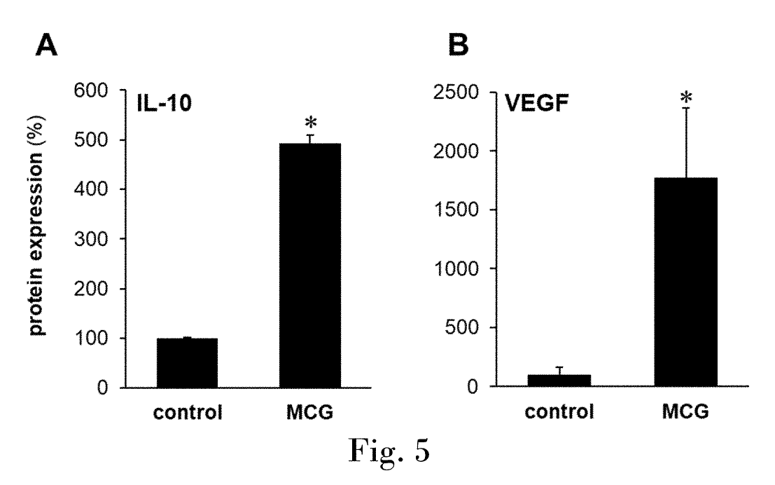

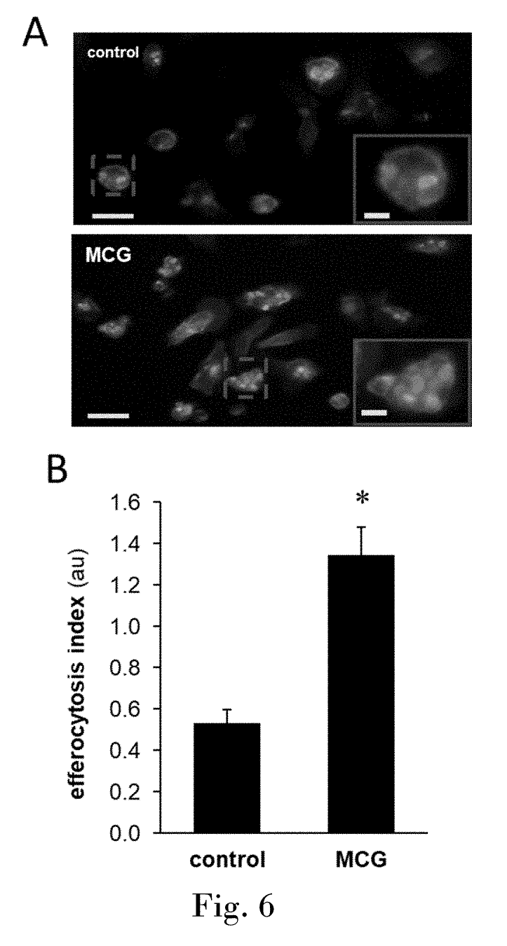

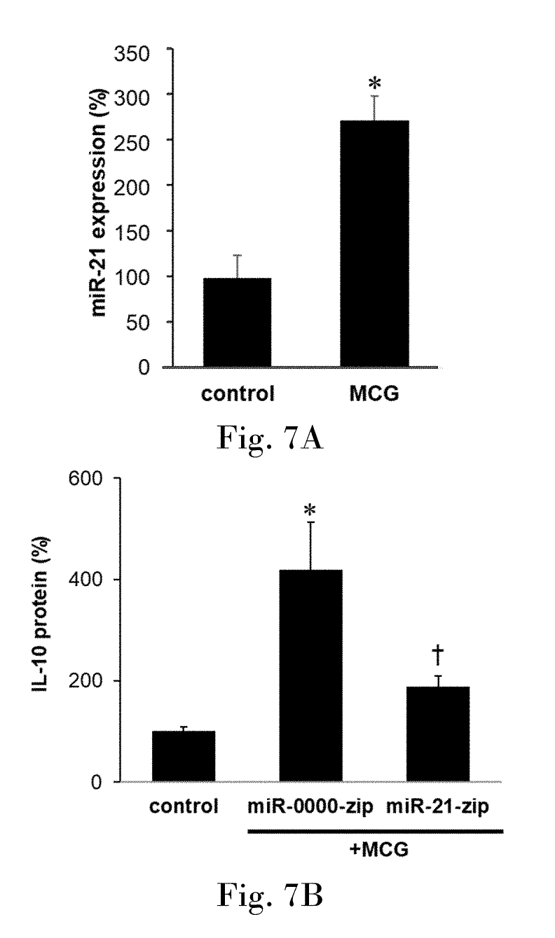

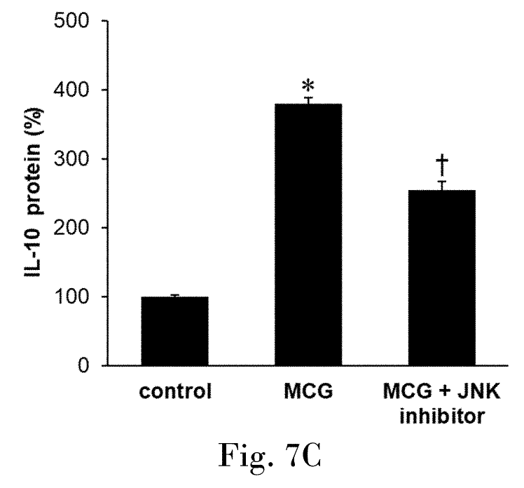

Increased apoptotic cell burden at wound-site exaggerates sustained inflammation at the wound-site. We have reported that engulfment of apoptotic cells by macrophages (aka, efferocytosis) is a signaling cue that drives polarization of M1 macrophages to M2 via miR-21-PDCD4-IL-10 pathway. The effect of MCG treatment on macrophage efferocytosis activity was determined. A significantly increased efferocytosis index was noted in macrophage treated with MCG as compared to matched untreated controls (FIGS. 6A & 6B). We have reported that successful efferocytosis results in induction of miR-21 expression that via PTEN and PDCD4 downregulation switches macrophage to an anti-inflammatory M2 phenotype. Concomitant to improved efferocytosis, a significant induction in miR-21 expression in MCG treated wound cells was observed (FIG. 7A). Using the THP-1 macrophage cells with miR-21 knockdown (miR-21-zip), we further demonstrate that MCG mediated induction of IL-10 in macrophages was miR-21 dependent (FIG. 7B) suggesting a central role for miR-21 in MCG-mediated resolution of inflammation. Our studies have demonstrated a key role of PDCD4-JNK-AP1 pathway in miR-21 mediated upregulation of IL-10. Pharmacological inhibition of JNK in THP-1 macrophages resulted in attenuated IL-10 production by MCG, indicating a role of miR-21-JNK pathway in MCG-mediated IL-10 release in macrophages (FIG. 7C).

### Discussion

A major component of the connective tissue, collagen has been recognized to induce signal transduction which in turn modulates several physiological functions like cell adhesion and migration, hemostasis, and immune function. Uptake of degraded collagen or collagen peptides at a wound-site is readily phagocytosed by macrophages. Whether such engulfment of collagen peptides induces any cellular signaling in macrophages remains unknown. The data herein evidence that a modified collagen based wound dressing composed of short and long chain peptides of collagen induces M2-like polarization in wound macrophages including production of copious amounts of anti-inflammatory and proangiogenic response by these cells. Parallel to findings of the current study, an increased macrophage infiltration in excisional wounds treated with modified collagen gel in a porcine model was also noted suggesting that

MCG possess macrophages chemoattractant property. GC-MS/MS studies from our laboratory characterized the structure of the MCG that is composed of long and short chain peptides derived from collagen. The mechanism of collagen peptide mediated macrophage chemoattractant function appears to be from promotion of production of MCP-1, a potent macrophage chemo-attractant, in the wounds and thereby increasing macrophage infiltration. At the wound-site, macrophages are known to exist in functionally distinct roles including the classical (proinflammatory, M1) and alternative (anti-inflammatory, prohealing, M2) activation states. While the pro-inflammatory M1 macrophages performs the clearing of infectious agents the M2 macrophages are more reparative in nature and aids in timely resolution of inflammation and promote angiogenesis. Chronic diabetic ulcers with unresolved inflammation display aberrant M1:M2 macrophage ratio and an imbalance between pro- and anti-inflammatory environment. CD40, CD16-32, CD11c and TLR4 are makers of M1 macrophage polarization while Dectin, CD163 and CD23 markers are expressed by M2 macrophages. Functional wound macrophages treated with MCG in vivo displayed a decrease in M1 macrophage polarization at the early inflammatory phases while an induction in the reparative M2 polarization phenotype was noted in the late inflammatory phases suggesting a shift in the wound macrophage polarization from M1 to M2. The shift in the phenotype of the wound macrophages is coupled with induction of the anti-inflammatory cytokine IL-10 and pro-angiogenic VEGF. This data is consistent with increased IL-10, Mrc-1 and CCR2 expression in MCG treated wounds. Concomitant to increased M2 macrophage polarization, an increased wound angiogenesis was noted in these wounds treated with MCG. Given that anti-inflammatory tissue mϕ0 have been directly implicated in angiogenesis it is plausible that the MCG-induced M2 polarization of macrophages promotes wound angiogenesis.

Mechanism of macrophage polarization includes complex interplay of multiple signaling pathways and transcription factors. This study identified that miR-21 plays a central role in MCG-induced macrophage polarization. We have recently underscored a major role of efferocytosis and microRNA-21 (miR-21) in macrophage transition from a M1 to an anti-inflammatory M2 phenotype featuring decreased TNF-α and increased IL-10. Efferocytosis or successful engulfment of apoptotic cells is known to promote an anti-inflammatory response in macrophages including induction of miR-21 expression. An impairment of efferocytosis in diabetic wounds led to unresolved inflammation. miR-21 promoted anti-inflammatory M2 like response in human macrophages by directly targeting Phosphatase and tensin homolog (PTEN) and Programmed cell death protein 4 (PDCD4) that subsequently inhibited NF-κB→TNFα or promoted JNK→AP-1→IL-10 production. Blocking of JNK resulted in an attenuation of MCG-induced IL-10 production suggesting that the anti-inflammatory effects of MCG involves miR-21 targeting PDCD4 followed by activation of JNK→AP-1→IL-10 pathway.

Collagen based wound dressing have been widely used in effective treatment of chronic wounds. The current understanding of the mechanisms of action of these dressings include i) serving as a substrate for high matrix metalloproteinase (MMP) in chronic wound environment; ii) the chemotactic property of the collagen breakdown products for cells critical in formation of granulation tissue and iii) the high absorbing nature helps in exudate management and maintaining a moist wound environment. This study identified and characterized a novel mechanism of action of collagen based wound dressings in modifying wound inflammatory response elicited by macrophages. MCG promoted an anti-inflammatory proangiogenic M2-like macrophage phenotype via miR-21-JNK mediated signaling pathway. The findings of this work provide a novel paradigm in macrophage-ECM interactions as well as reshape the understanding of the mechanisms of action of collagen based dressings in the treatment of chronic wounds, and implications for use of such compositions in the treatment of a wide variety of conditions implicated in the JNK pathway.

1. A method of modulating JNK activity in a cell expressing JNK, comprising contacting the cell with a therapeutically effective amount of a therapeutic modified collagen.

2. The method of claim 1, wherein said modified collagen is a hydrolyzed bovine collagen.

3. The method of claim 2, wherein said collagen comprises an amount of each of Type I, Type II, and Type III collagen, wherein the amount of Type I collagen is greater than the amount of Type II or Type III collagen, and wherein the amount of Type III collagen is greater than the amount of Type II collagen.

4. The method of claim 2, said modified collagen further comprising hemoglobin and/or carbonic anhydrase II.

5. The method of claim 2, wherein said modified collagen is a collagen gel comprising modified collagen of long and short polypeptides, dispersed in an aqueous matrix comprising water and glycerine.

6. The method of claim 5, comprising from about 25 to about 75% by weight modified collagen dispersed in said aqueous matrix, based upon the total weight of the gel composition taken as 100% by weight.

7. The method of claim 2, wherein said modified collagen comprises a plurality of proteins characterized by Table 1.

8. The method of claim 1, wherein said cell is a healthy cell.

9. The method of claim 8, wherein said cell is a macrophage.

10. A method of modulating JNK activity in a patient in need thereof, said method comprising:
administering to the patient a therapeutically effective amount of a therapeutic modified collagen composition to said patient,
wherein said modified collagen increases JNK expression in said patient.

11. The method of claim 10, wherein said therapeutic modified collagen promotes efferocytosis of apoptotic cells in said patient.

12. The method of claim 10, wherein said increase in JNK expression treats JNK-mediated disorder is caused by under-expression of JNK.

13. The method of claim 12, wherein said disorder is an inflammatory, autoimmune, cardiovascular, ischemic, neurodegenerative, or metabolic condition, infection, diabetes, or cancer.

14. The method of claim 10, wherein said modified collagen is a hydrolyzed bovine collagen.

15. The method of claim 14, wherein said collagen comprises an amount of each of Type I, Type II, and Type III collagen, wherein the amount of Type I collagen is greater than the amount of Type II or Type III collagen, and wherein the amount of Type III collagen is greater than the amount of Type II collagen.

16. The method of claim 14, said modified collagen further comprising hemoglobin and/or carbonic anhydrase II.

17. The method of claim 14, wherein said modified collagen comprises a plurality of proteins characterized by Table 1.

18. The method of claim 10, wherein said administering comprises externally applying said therapeutic modified collagen composition to said patient or injecting said therapeutic modified collagen composition into said patient.

19. A JNK modulator comprising a therapeutic modified collagen, said collagen comprising a plurality of proteins characterized by Table 1.

20. A method of stimulating efferocytosis comprising, administering to a subject in need thereof a therapeutically effective amount of a JNK modulator according to claim 19.
